(19) United States
(12) Patent Application Publication
(10) Pub. No.: US 2008/0176199 A1
(43) Pub. Date: Jul. 24, 2008

(54) Prompting System For CPR Delivery

(75) Inventors: Ronald E. Stickney, Edmonds, WA (US); Robert E. Smith, Lynnwood, WA (US); James M. Owen, Redmond, WA (US); Alidene Doherty, Issaquah, WA (US)

Correspondence Address: MARY Y. REDMAN;MEDTRONIC, INC., 710 MEDTRONIC PARKWAY NE M.S. LC340, MINNEAPOLIS, MN, 55432-5604, US

(73) Assignee: PHYSIO-CONTROL, INC., Redmond, WA (US)

(21) Appl. No.: 11/971,877

(22) Filed: Jan. 9, 2008

Related U.S. Application Data
(60) Provisional application No. 60/880,219, filed on Jan. 11, 2007.

(51) Int. Cl. G09B 23/28; A61B 5/02; A61B 5/04

(52) U.S. Cl. 434/265; 600/508; 600/509

(57) ABSTRACT

A method of providing instruction on the performance of chest compressions includes providing a series of signals of a first type corresponding to the desired rhythm of delivery of chest compressions in a chest compression series, and providing signals of a second type which indicate a desired point in the first series. The desired point may be a point near the end of the chest compression series. The signals of the second type may be a voiced countdown to the end of the compression series. The signals of the first type may be a series of identical sounds delivered in the desired rhythm for chest compressions, and the signals of the second type may be sounds distinct from those of the first type which correspond to the rhythm of the last N compressions in the series. The desired point in the first series may include a first point at a desired interval from the first compression, where the interval is measured in number of compressions or elapsed time. A protocol may be chosen between a protocol for a patient with a secured airway and one for a patient with an unsecured airway.

### BACKGROUND OF THE INVENTION

1. Field of the Invention

The present invention relates generally to CPR prompting and instruction. More particularly, the present invention relates to a method and apparatus for providing timing signals to a rescuer who is performing CPR.

2. Technical Background

CPR, or cardio-pulmonary resuscitation, is a technique used in resuscitation of a patient in a cardiac emergency. CPR is performed by applying sequential compressions to the chest of a patient in order to affect blood flow to vital organs. CPR guidelines by the American Heart Association also call for periodic ventilation of the patient. Proper performance of the chest compressions and ventilations will enhance the patient's chance of survival. Attributes of proper performance include the rate at which chest compressions are given, the number of chest compressions in a sequence or the time duration of a sequence of chest compressions, the frequency and rate of ventilations, and the time duration of each single ventilation.

Common CPR protocols typically involve the delivery of a series of chest compressions, usually followed by a series of ventilations (in some protocols the ventilation series may include only a single ventilation). For example, a commonly used protocol for adults is thirty chest compressions followed by two ventilations and a commonly used protocol for infants is 15 compressions followed by two ventilations. Some CPR protocols may define a chest compression series in terms of the length of the time interval over which compressions are delivered. In some CPR protocols, the ventilation series may overlap With the compression series. For example, a typical CPR protocol for patients with an airway secured by an endotracheal tube or other device is other is continuous chest compressions for a given time period with one ventilation given every 6 to 8 seconds (with no pause in chest compressions). Desired protocols for delivery of CPR may vary depending on factors such as age classification of the patient (i.e., adult or child/infant), patient airway status (for example, whether the patient has his airway secured by intubation), whether the CPR is being delivered by one or two persons, or whether the person delivering CPR is a medical professional or a layperson

The American Heart Association is a source of guidelines on CPR protocols, including the rate at which chest compressions should be delivered (for example, 100 compressions per minute) and the time over which ventilation should be provided (for example, each ventilation should have duration of about one second). According to “2005 American Heart Association Guidelines for Cardiopulmonary Resuscitation and Emergency Cardiovascular Care”, Circulation, Volume 112, Issue 24 Supplement; (Dec. 13, 2005), which is incorporated by reference herein, rescuers should minimize interruptions in chest compressions.

### SUMMARY OF THE INVENTION

A method of providing instruction on the performance of chest compressions includes the steps of providing a series of signals of a first type corresponding to the desired rhythm of delivery of chest compressions in a chest compression series; and providing a signal of a second type which indicates a desired point in the first series.

In this method, the desired point may be a point near the end of the chest compression series. The signal of the second type may be a voiced countdown to the end of the chest compression series. The signals of the first type may be a series of identical sounds delivered in the rhythm desired for the chest compressions and the signal of the second type is a second series of sounds which are distinct from the sounds of the first series and which correspond to the rhythm of the last N compressions in the first series, where N is a predetermined number. N may be greater than or equal to two.

The voiced countdown may includes the words “two, one” in a rhythm corresponding to the desired rhythm of the last two compression of the series.

The desired point in the first series may include a first point at a desired interval from the first compression. The method may further include providing a signal at a second point in the first series at a desired interval from the first point. The desired interval may be measured in number of compressions. The desired interval may be measured in time.

The signals of the first the may be tonal signals of a first type, and the signal of the second type may be a series of a second kind of tonal signals which are distinguishable from the tonal signals of the first type. The method may further include providing a prompt which instructs the user to provide ventilation to the patient, the duration of the prompt being at least as long as the desired duration of the ventilation. The ventilation prompt may include a voice prompt. The ventilation prompt may be a sound prompt which approximates the sound of a ventilation bag.

The method may further include the step of choosing between a first prompting protocol appropriate for a patient with a protected airway and a second prompting protocol appropriate for a patient with an unprotected airway.

The method may further include the step, prior to the step of providing a series of signals of a first type, of detecting delivery of a chest compression, and commencing delivery of the prompts in response to detecting a chest compression.

A method of instructing on delivery of CPR to a patient includes the steps of: choosing between a protocol for a patient with a secured airway and a patient with an unsecured airway; delivering prompts according to the chosen protocol which include rhythmic prompts delivered at the desired rate of chest compressions.

The choosing step may further include choosing between a protocol for an adult patient and a protocol for a non-adult patient.

The step of choosing may be performed during the delivery of a series of chest compressions.

The method may further include the step of providing a second series of prompts prior to completion of the chest compression series which provides indication that the end of the chest compression series is nearing.

The method may further include the step, prior to the step of delivering prompts, of detecting delivery of a chest compression, and commencing delivery of the prompts in response to detecting a chest compression.

A device for providing instruction on the performance of chest compressions may include a user interface output device; and a processor capable of instructing the user interface output device to produce a series of signals of a first type corresponding to the desired rhythm of delivery of chest compressions in a chest compression series and a signal of a second type which indicates a desired point in the first series.

The device may further include a sensor in communication with the processor that detects a parameter indicative of delivery of a chest compression. The parameter may be patient impedance. The sensor may include electrodes adapted to be applied to a patient, and the device may further include an energy storage device electrically coupled to the electrodes.

The device may further include a memory in which instructions for a plurality of CPR protocols is stored; and a user interface input in communication with the processor; wherein the processor is capable of calling up a CPR protocol from the memory in response to the input information.

It is to be understood that both the foregoing general description and the following detailed description are exemplary and explanatory and are intended to provide further explanation of the invention as claimed. The accompanying drawings are included to provide a further understanding of the invention and are incorporated in and constitute a part of this specification, illustrate several embodiments of the invention and, together with the description, serve to explain the principles of the invention.

### BRIEF DESCRIPTION OF THE DRAWINGS

FIG. 1 is a block diagram of a metronome system;

FIG. 2 is a block diagram of an external defibrillator system into which a system is integrated; and

FIG. 3 is flow chart of a process that may be performed by the defibrillator of FIG. 2.

FIG. 4 is an illustration of a defibrillator/monitor.

FIGS. 5 through 8 are examples of screens that may be displayed when t with a defibrillator/monitor operating in a manual mode.

FIGS. 9 through 14 are examples of screens that may be displayed when an embodiment with a defibrillator/monitor operating in an AED mode, or with an AED having a display screen.

### DETAILED DESCRIPTION

A metronome prompting system provides rhythmic signals to guide the user in pacing and timing of chest compressions and, in some embodiments, also provides signals to guide in the pacing and timing of ventilations. As used herein, a metronome or metronome system delivers a rhythmic signal (such as a repeated sound or flashing light) at a rate corresponding to the desired rate for chest compression, and may also deliver other signals or prompts. The metronome system may provide a series of signals of a first type corresponding to the desired rhythm of delivery of chest compressions in a chest compression series; and signal of a second type which indicates a desired point in the first series.

The desired point may be a point near the end of the chest compression series, to alert the rescuer that a change from chest compressions to ventilations is nearing. In the transition from a series of compressions to ventilations, time can be lost as the rescuer(s) transitions physically and mentally from the task of delivering compressions to the task of delivering ventilations. This is true in circumstances both where there are two rescuers and also for a single rescuer.

Alternatively, it may be a point elsewhere in the series of chest compressions (for example, a midpoint), to keep the rescuer apprised of how far he has progressed through the chest compression series and/or how much longer (in time or number of compressions) the chest compression series will continue. The metronome system may include a user input though which a user may choose a CPR protocol to be followed.

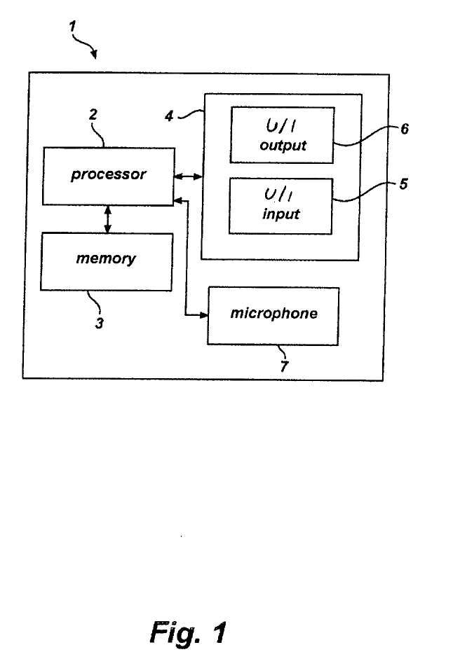

Referring to FIG. 1, a stand-alone metronome includes a processor 2 which interfaces with a memory 3 in which various CPR protocols are stored. A user interface 4 includes a U/I input device 5 through which the user inputs information which will affect what CPR protocol is used or the user's choice of CPR protocols. These may include one or more of the following: whether the patient is an adult or a child/infant, whether or not the patient's airway is secured (for example, via incubation), whether CPR will be administered by one or by two providers, or other variables which may influence choice of CPR protocol. The user interface 4 also includes a U/I output device 6 which provides visual or aural signals to the user. The processor receives the user input, calls the corresponding CPR protocol out of the memory and instructs the u/i output 6 to provide the appropriate signals and prompts.

Processor 2 may take the form of a microprocessor, digital signal processor (DSP), ASIC, FPGA, or other logic circuitry programmed or otherwise configured to operate as described herein. Memory 3 may include any of a variety of electrical, magnetic or optical media, such as a RAM, ROM, CD-ROM, EEPROM, or magnetic disk. User input 5 may include input devices such as a keypad, selector buttons, toggle switches, selector dials, or touchscreen soft keys, Output devices may include indicator lights, a CRT, LED, or LCD screen, and a speaker.

The stand-alone metronome system 1 may be contained in a compact housing having a size and shape which make it suitable to be hand-carried to a patient and placed near the user(s). The metronome 1 may be powered by replaceable and/or rechargeable batteries.

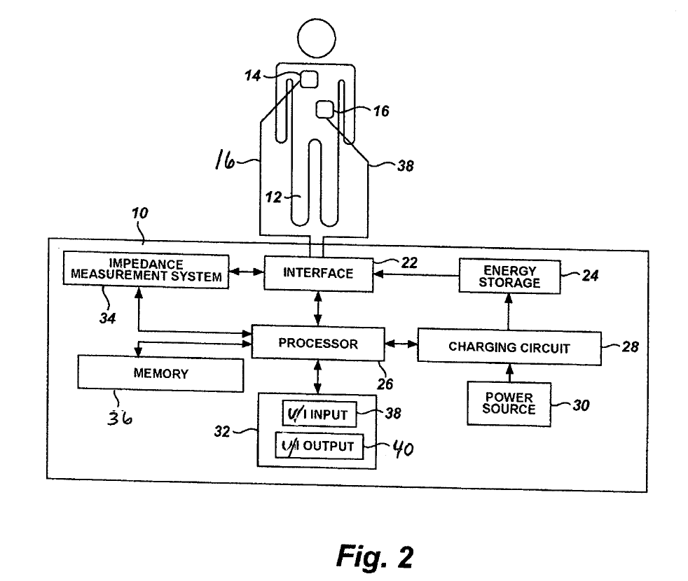

Referring now to FIG. 2, a metronome may be integrated into an emergency medical device such as an external defibrillator 10 which in use is coupled to a patient 12. Examples of external defibrillators into which a metronome could be embedded include those sold under the LIFEPAK brand by Physio-Control, Inc. of Redmond, Wash. FIG. 2 is a block diagram of a typical external defibrillator. Defibrillator 10, which may be a manual defibrillator or an automated external defibrillator (AED), delivers defibrillation pulses to patient 12 via electrodes 14 and 16, which may be adhesive electrode pads placed on the skin of patient 12. Electrodes 14 and 16 are coupled to defibrillator 10 via conductors 18 and 20 and interface 22. In a typical application, interface 22 includes a receptacle, and conductors 18 and 20 plug into the receptacle. Interface 22 includes a switch (not shown) that, when activated, couples an energy storage circuit 24 to electrodes 14 and 16. Energy storage circuit 24 includes components, such one or more capacitors, which store the energy to be delivered to patient 12 via electrodes 14 and 16 as a defibrillation pulse. Before a defibrillation pulse is delivered to patient 12, a processor 26 directs a charging circuit 28 to charge energy storage circuit 24 from a power source 30.

Processor 26 may take the form of a microprocessor, digital signal processor (DSP), ASIC, FPGA, or other logic circuit programmed or otherwise configured to operate as described herein. Charging circuit 28 comprises, for example, a flyback charger that transfers energy from a power source 30 to energy storage circuit 24. Power source 30 may comprise, for example, batteries and/or an adapter to an exterior power source such as an electrical outlet.

Electrodes coupled to the patient 12 sense ECG signals in the heart, which are communicated to the processor via conductors 18 and 20 and interface 22. The processor analyses these ECG signals and determines whether a defibrillation shock or CPR is appropriate therapy. Examples of algorithms and analysis processes for determining if defibrillation shock or CPR therapy is appropriate may be found in the commercially available defibrillators mentioned above.

Memory 36 may include program instructions that cause processor 26 to perform the analysis, and to perform the other functions ascribed to processor 26 herein. Memory 36 may include any of a variety of electrical, magnetic or optical media, such as a RAM, ROM, CD-ROM, EEPROM, or magnetic disk.

Besides detecting and analyzing ECG signals, the defibrillator 10 may detect patient impedance by any of several known impedance measurement techniques to measure the transthoracic impedance of patient 12. For example, a low-level current technique may be used to measure the impedance. In this technique, an impedance measurement system 34 employs a current source (not shown) to generate an “excitation current,” also called a “carrier,” that is applied to patient 12 through interface 22 and electrodes 14 and 16. The excitation current may be an alternating current signal of known magnitude and frequency. The excitation current is much smaller in magnitude than a typical defibrillation current delivered during delivery of a defibrillation shock. A typical excitation current has a magnitude of around 100 microamperes. The frequency of the excitation current is generally within a range from 5-100 khz, and may be approximately 62 kHz. Impedance measurement system 34 may detect the response to the excitation current as a time-varying voltage difference between electrodes 14 and 16. System 34 may include amplifiers, filters, and the like (not shown) to detect the voltage difference and process the resulting signal, and an analog-to-digital filter (not shown) to convert the signal to a digital signal. A controller (not shown) of system 34 that is responsive to signals received from processor 26 may control the current source, measure the magnitude and phase of the voltage difference in order to measure the impedance of patient 12, and provide the measured transthoracic impedance to processor 26. Alternatively, the controller may be embodied within processor 26. Since a compression of the chest will change the impedance of the patient, the impedance measurement can be used to detect a chest compression.

The defibrillator 10 has a user interface 32. The user interface includes a U/I input 38 through which the user inputs information which will affect what CPR protocol is used. These may include one or more of the following: whether the patient is an adult or a child/infant whether or not the patient's airway is secured (for example, via intubation), whether CPR will be administered by one or by two providers, or other variables which may influence choice of CPR protocol. The user interface also includes a U/I output 40 which provides visual or aural signals to the user. When the ECG analysis indicates that CPR is called for, the processor receives the user input, calls the CPR protocol corresponding to the inputted information out of the memory and instructs the U/I output 6 to provide the appropriate signals and prompts.

This user interface includes a U/I input 38 which communicates with the processor 28 and a U/I output 40 which receives commands from the processor 28. The user interface of the defibrillator may be used in the manner described above and perform the functions described above for a The U/I input may be used to input information which will affect what CPR protocol is used. These may include one or more of the following: whether the patient is an adult or a child/infant, whether or not the patient's airway is secured (for example, via intubation), whether CPR will be administered by one or by two providers, or other variables which may influence choice of CPR protocol. The user interface 4 also includes a U/I output device 6 which provides visual or aural signals to the user. The processor receives the user input, calls the corresponding CPR protocol out of the memory and instructs the U/I output 6 to provide the appropriate signals and prompts.

For the stand-alone metronome and the metronome integrated into an emergency medical device, the metronome signals may be visual (such as flashing lights or graphics on a display screen), or may he aural. Preferably, at least three types of signals will be delivered to the user. These will include a first type of signal for chest compressions, a second type of signal for ventilations, and a third type of signals to indicate an upcoming transition from compressions to ventilations (or, in a protocol where ventilations are given without a pause in compressions, to indicate an upcoming ventilation series). Preferably, all three signal types will be distinguishable form one another by the user. Where flashing lights are used, different colors may distinguish between compressions, transitions and ventilations. The aural signals may be any of a variety of sounds such as tones, beeps, tocks, clicks, and the like, or may be voiced (for example, “press-press-press” for compressions, “ventilate” or “blow” for ventilations). In an embodiment, a user may choose whether to have the metronome deliver voiced signals or non-voiced sounds (for example, tones, beeps, clicks, tocks, or other non-verbal sounds) through a set-up menu upon device set-up.

The signals for chest compressions will be rhythmic signals such as a series of identical sounds delivered at a rate corresponding to the desired rate for chest compressions. A sound that is suggestive of or approximates the sound of ventilation (the “hiss” of an AMBU bag when squeezed, for example) may be used for a ventilation signal. The sound signal used for each ventilation may have a duration that corresponds to the desired duration of the ventilation. For a ventilation series having more than one ventilation, the ventilation sound signals will also be delivered at a rate equal to the desired rate for ventilation delivery.

The transition signals will advise the user or users that a transition from chest compressions to ventilations (with or without a pause in chest compressions) is coming up soon. For example, where tones, beeps, clicks or tocks are used to indicate chest compressions in a 30 compressions ventilations protocol, the transition signal may be a voiced countdown of the last few compressions in a series. The last six compressions in an example where a ‘tock’ sound is used for compressions may be signaled as:
tock-tock-tock-“three-two-one”,
   or as:
tock-tock-tock-tock-“two-one”.

If a voiced “ventilate” is used as the ventilation signals, this would be followed by “Ventilate. Ventilate”, giving the following series of prompts:
tock-tock-tock-“tree-two-one. Ventilate. Ventilate”,
   or
tock-tock-tock-tock-“two-one. Ventilate. Ventilate”.

The stand-alone metronome and the metronome integrated with a defibrillator may optionally include a mechanism for maintaining the apparent Sound Pressure Level (SPL) at a given distance from the device at a desired level, to optimize intelligibility of the aural signals. For example, SPL at a one meter distance from the device may be maintained at approximately 10 dB, C weighted, slow averaged, SPL above the ambient background noise. This can be done by periodically or continuously sample the background ambient noise with a microphone (see FIG. 1, no. 7) and necessary signal conditioning by processor 2 to measure the SPL. With the prompt playback system characterized, the playback SPL at one meter will be known for a given amplifier power. Based on the measured SPL, the amplifier power can be adjusted to achieve a selectable constant between 6 and 12 dB, C weighted, slow averaged, SPL above the ambient noise. This prompt volume may be periodically or continuously adjusted to maintain the selected constant between 6 and 12 dB signal to noise.

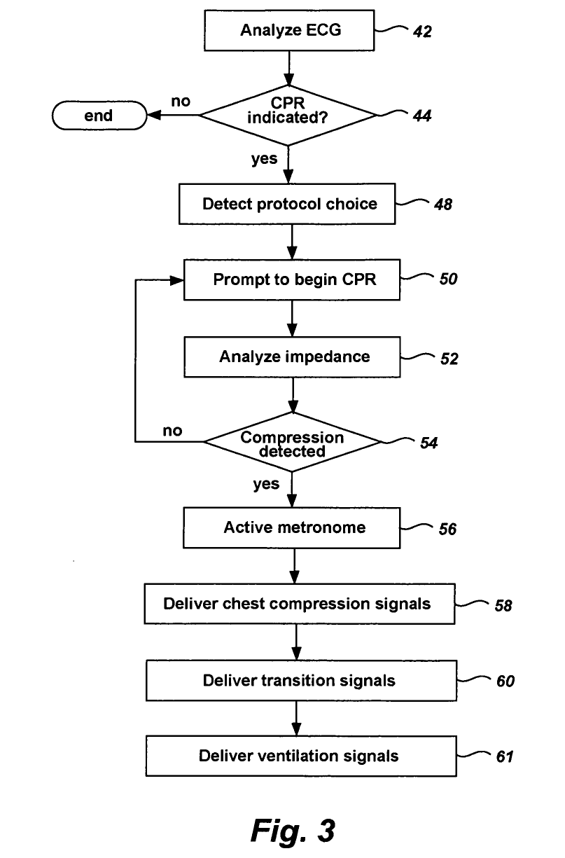

Referring now to FIG. 3, an example of a process that may be employed by the external defibrillator 10 of FIG. 2 for CPR prompting is illustrated. The processor analyses ECG signals and any other factors used by the defibrillator to determine if CPR should be prompted for (block 42). At block 44, if CPR is not indicated, then the CPR prompting process is ended and defibrillator 10 continues operation with a non-CPR process (for example, prompting for delivery of a shock if a shockable heart rhythm was detected). If CPR is indicted, processor 26 retrieves from memory 38 the CPR protocol instruction choice which corresponds to previously input information (block 48). The processor then controls the user interface 32 to provide an indication to the operator of defibrillator 10 that CPR should be administered, such as an indicator light, graphics or text on an LCD screen, or a voice prompt. The voice prompt may be a prompt like “Start CPR”, and may advise the user to follow the rhythm of the metronome signals. At this point, the metronome signals could be initiated immediately after delivery of the preceding prompt or at a preset time interval after the preceding prompt.

Alternatively, as in the process illustrated in FIG. 3, the metronome chest compression signals may be initiated upon detection of the user administering a chest compression. Patient impedance may be analyzed (block 52) in the manner described above to detect a chest compression. Other mechanisms and devices for detecting a chest compression may be used. Once a chest compression is detected (block 54), the metronome signals will be activated (block 56). If no chest compression is detected after some period of elapsed time (due, for example, to user error), the U/I output may again prompt to begin CPR (block 50), and the impedance analysis and compression detection steps may be repeated. If again no chest compression is detected after the designated time period, the process may again return to the step of prompting for the start of CPR. This may be repeated a desired number of times or for a desired number of seconds after which, if chest compressions are still not detected, the process of FIG. 3 exits to a non-CPR procedure. There may be instances where a user is deliberately choosing to not deliver CPR. For example, a medical professional may deem an alternative therapy to be called for, or there may be other reasons why CPR is not being administered. To accommodate such situations, the user interface 32 may provide a mechanism for the user to abort the process of FIG. 3. This mechanism may be, for example, a soft key on a touchscreen indicating “CPR metronome off”, as an alternative to waiting for the FIG. 3 process to time out.

Returning to block 54, if a compression is detected, the metronome sisals will be activated (block 56). A series of chest compression signals will be delivered at a rhythm desired for chest compression delivery. These will be followed by transition signals, such as the countdown signals discussed above. The transition signals will be followed by ventilation signals, as discussed above.

Although the metronome has been described in terms of signaling for chest compressions and for ventilations, there may be conditions under which signals for ventilations are not desired. A compressions-only protocol may be one of the protocols stored in memory. If user input indicates a compressions-only signaling protocol is to be delivered, then transition signals may be used to indicate the end of the chest compression series, giving the user an indication that transition to a new action (such as, for example, removing hands from the patient while an ECG analysis is done) is approaching.

There may be situations where it is desired that the CPR protocol choice be changed during delivery of CPR or at some other point during the resuscitation event. For example, if a single lay rescuer begins CPR and then medical professionals arrive, they may want to change from a protocol appropriate for a single lay user to another protocol. Or, if a patient with an unsecured airway is intubated so as to secure the airway, the users may wish to change the choice of CPR protocols. The processor 26 may check for CPR protocol input before the initial series of chest compressions and then check again during the series or before each additional series to see if there has been a change in protocol choice.

User input concerning CPR protocol choice may be indicated in a direct manner or in an indirect manner. For example, the user interface may display buttons, dial settings, or soft keys for “Adult” and “infant/child”, or for “secured airways and unsecured airway” with the user inputting the choice of age classifications and airway status. Alternatively, input on factors such as age classification may be derived from the processor from indirect input. For example, when using a defibrillator, a user may choose to connect adult electrodes or pediatric electrodes, or when using a manual defibrillator, may input a choice of defibrillation energy levels. The processor may receive information on which electrodes have been connected, or what energy level has been chosen, and use that to choose an adult or infant/child CPR protocol.

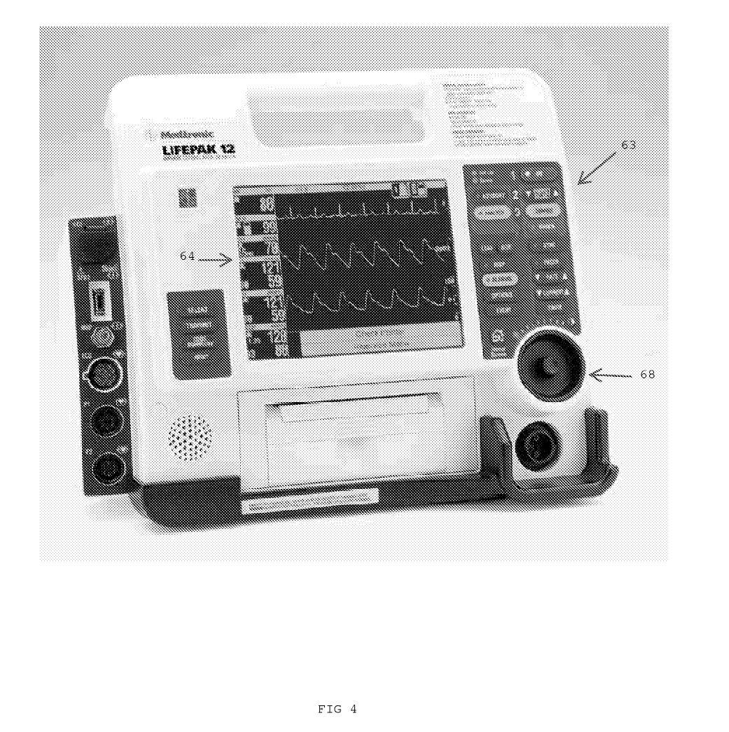

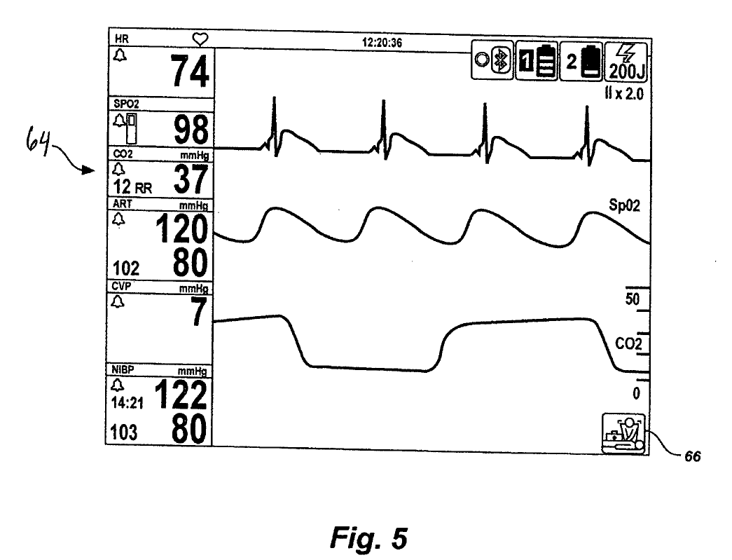

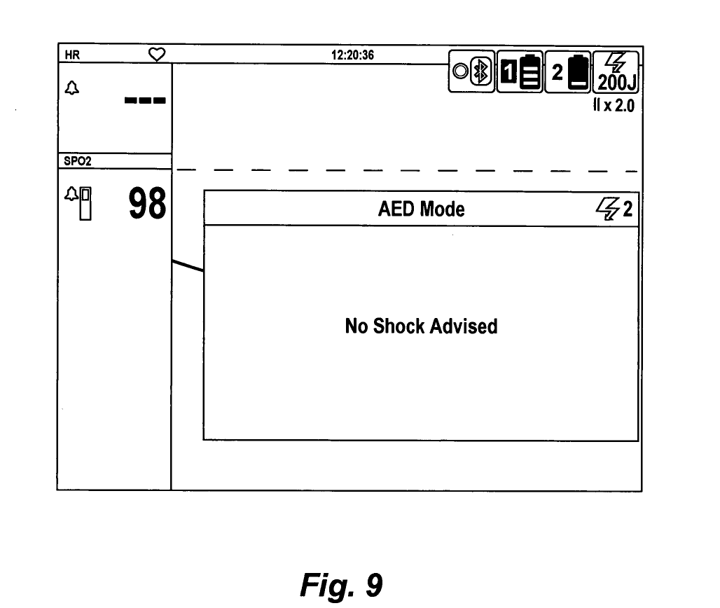

Referring now to FIG. 4, a defibrillator/monitor that 63 can be operated in a manual mode has a display screen 64 on which various vital signs may be displayed. Referring to FIGS. 5 through 14, examples of displays in a defibrillator/monitor in which an embodiment of the metronome is embedded will be described. FIGS. 5-8 illustrate display examples for a defibrillator operating in a manual mode; FIGS. 9 through 14 are examples of displays in a defibrillator/monitor operating in AED, or in an AED with a display screen

Referring now to FIGS. 5 through 8, when operating a defibrillator in the manual mode, a user may choose to initiate operation of the metronome at any time through a user input on the user interface. The user input may include a CPR icon 66 on the screen 64. The icon 66 may be a screen button on a touchscreen where the user can indicate a choice to activate the metronome by touching the icon, or, a hard key button may be provided adjacent the icon through which a user can indicate this choice. Or, other user input means such as a selector knob 68 like that found on the commercially available LIFEPAK® 12 defibrillator/monitor can be used.

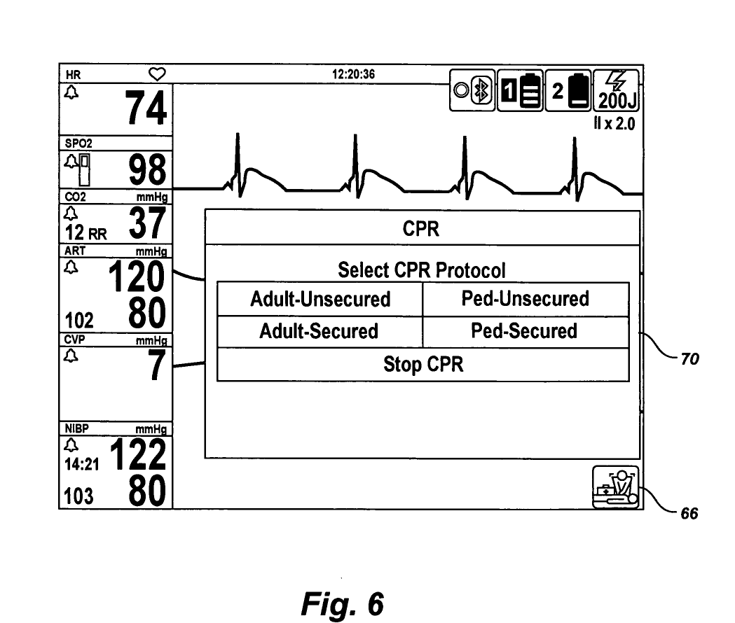

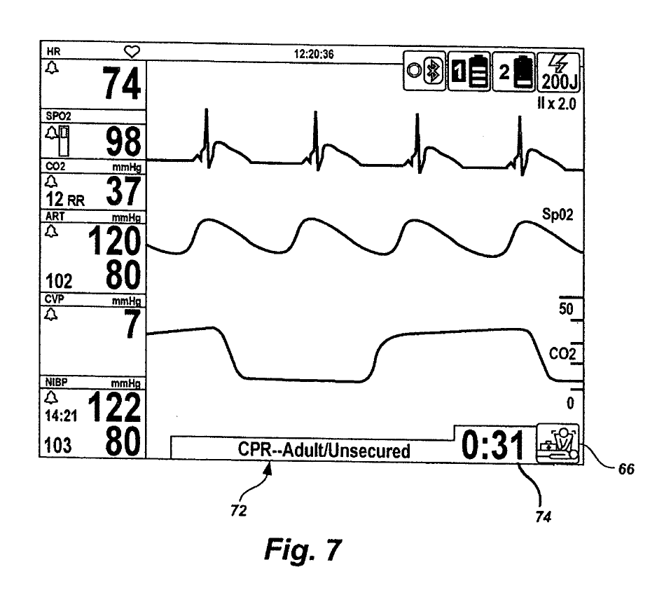

In the illustrated embodiment, once the user has chosen to activate the CPR metronome, a menu 70 (see FIG. 6) with choices of CPR protocols appears. The user indicates the choice of protocol through input means such as, for example, touchscreen buttons, a selector knob, or appropriately arranged hard keys. In one alternative, once the protocol choice is made, the metronome signals will commence and the screen will display the protocol being used 72 (see FIG. 7) and may also display the elapsed time 74 since the CPR metronome has been activated, which can serve as an approximation of time spent delivering CPR. In another alternative where the defibrillator senses a first chest compression as described above, the metronome signals will commence upon sensed delivery of the first chest compression and the timer on the display will display elapsed time since the first chest compression in the current CPR period, giving a more exact indication of time spent in CPR delivery. The time display may display the time already spent in the CPR period or alternatively, the time remaining in a CPR period. In another alternative, a count of the number of compressions performed or the number of compressions remaining to be performed may be displayed in place of or in addition to the time display.

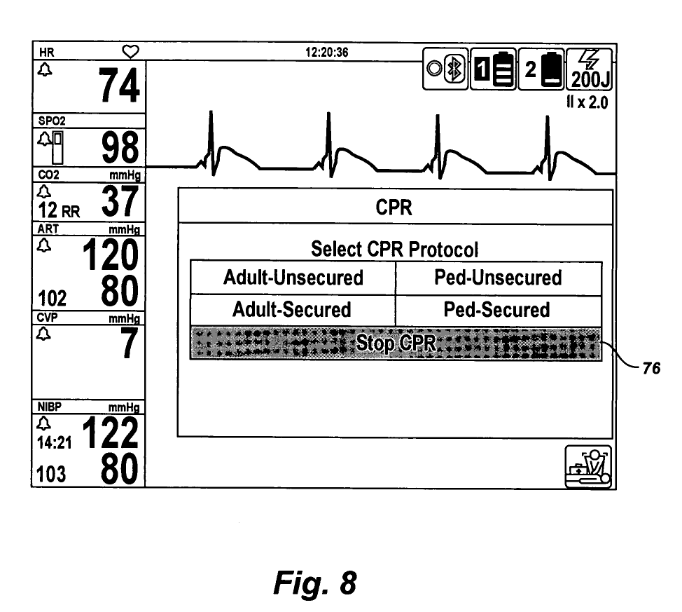

In the illustrated embodiment, the CPR metronome icon 66 remains on the screen during CPR delivery so that the user may reenter the CPR protocol menu at any time during CPR delivery to change from one protocol to another during CPR delivery, or to stop the metronome (see 76 in FIG. 8).

Were a new protocol is chosen, the instructions given by the processor to the CPR metronome output may cause it to pick up at the corresponding point in the newly chosen protocol. For example, if protocol choice is changed from “adult-unsecured airway” to “adult-secured airway” at a point one-third of the way through the adult-unsecured protocol, then the metronome would commence to signal the last two-thirds of the “adult-secured airway” protocol immediately after stopping the “adult-unsecured” signaling.

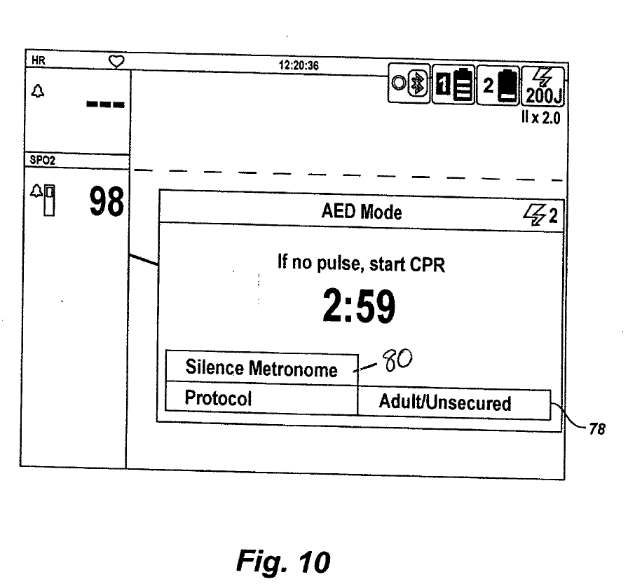

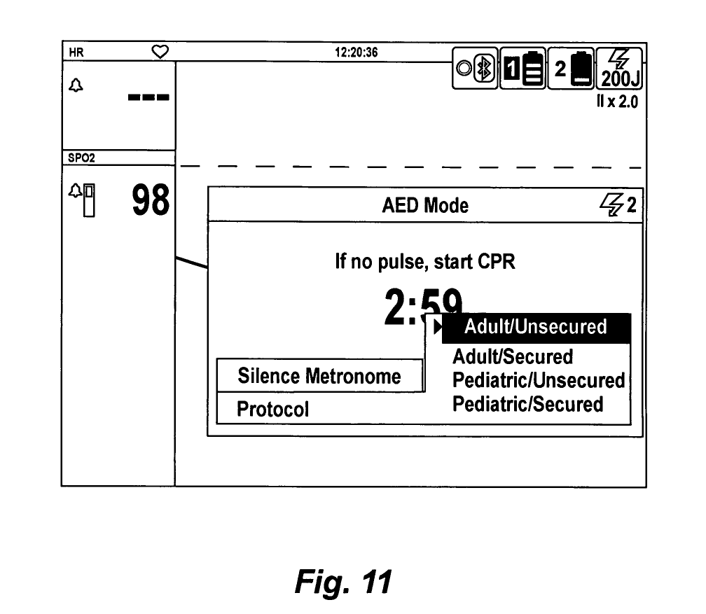

FIGS. 9 through 14 are examples of screen displays for an AED with a display screen or a defibrillator/monitor operated in AED mode in which the metronome is embedded. The screen displays may show text messages that parallel aural voice prompts. For example, in FIG. 9, where an ECG analysis indicates that no shock is advised, a text giving this information is displayed. In FIG. 10, a message instructing the user to start CPR if no pulse is found is displayed. In one alternative, a menu 78 allowing the user the choice of protocols or the choice of silencing the metronome is available. This may be desirable, for example, in a defibrillator/monitor used by professionals. Alternatively, the display could show only the prompt to start CPR, or the prompt plus the protocol menu, or only the prompt plus the “silence metronome” 80 menu choice. FIG. 11 shows the options available in the illustrated example when the protocol menu is chosen. As above, any other collection of protocol choices which the metronome can deliver may be displayed.

As seen in FIGS. 10 and 11, a clock on the screen may display time spent delivering CPR or the time remaining for CPR delivery, or a count-down of compressions to be delivered, or a count-up of compressions already delivered, may be displayed instead of or in addition to the time.

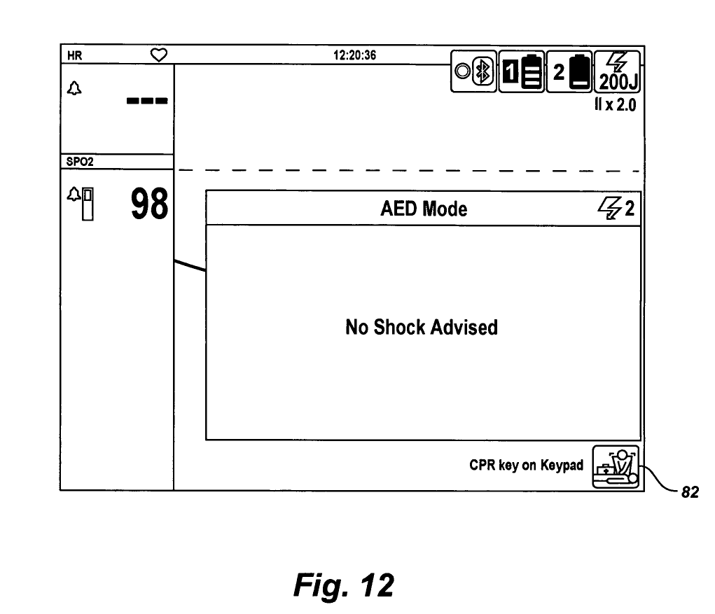

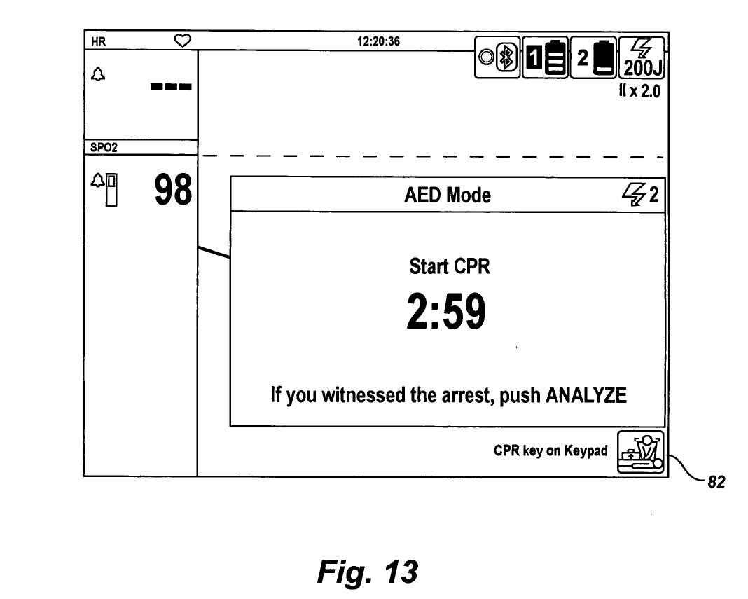

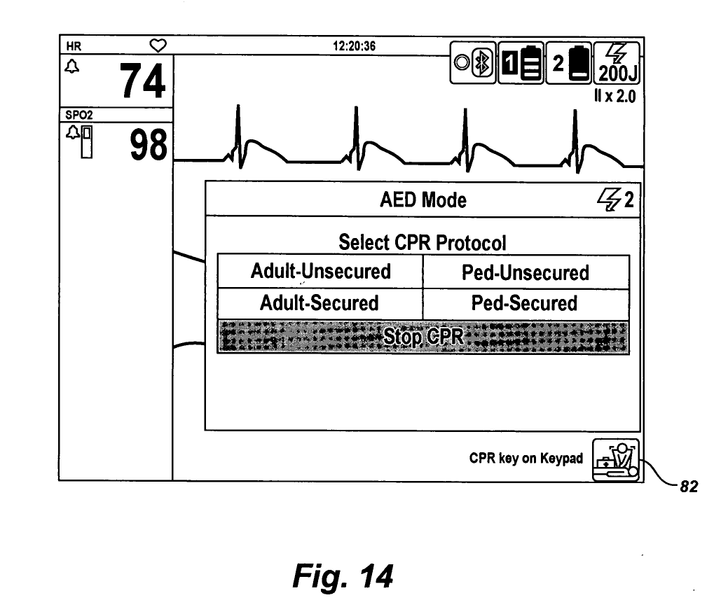

FIGS. 12 through 14 illustrate an example where, instead of a CPR icon on a screen, a CPR key or button 82 is provided on the device (for example, on a keypad) In one example, when the CPR button is pressed, the choice of protocol and/or the “silence metronome” (to silence the metronome sound signals) or “stop CPR” (to stop the metronome and exit CPR mode) options appear on the screen (see FIG. 14). In other embodiments, there may be other menu choices or metronome options that can be made to appear on the screen when the CPR button is pushed. FIG. 13 illustrates an example where the screen displays a request for user input on a condition that may affect the therapy or protocol to be delivered. US Published Patent Application No. 2006/0058848 entitled “AED with User Inputs in Response to Prompts” (filed Mar. 16, 2006) is hereby incorporated herein by reference in its entirety. This published patent application includes examples of questions and prompts for user input of information which may be incorporated into some embodiments if desired.

In a device with both a manual mode and an AED mode, where a CPR timer or compression count is displayed, it may be desired to have the counter count up the delivered compressions in one mode and count down how many remain in the other mode, and/or have a timer display time spent in CPR in one mode, and time remaining in the other mode. For example, a countdown of time remaining to be spent in CPR delivery could be displayed for AED mode, while a count-up of time spent delivering CPR could be displayed when in manual mode.

In embodiments where the option to silence the metronome is available to the user, the visual timer/counter could remain displayed while the metronome sound signals are silenced.

The processor may be programmed so that sounds made by the metronome have the desired priority over other audible prompts and alarms which delivered by the device. For example, the metronome signals may be given priority over all other prompts or signals during the period when CPR is delivered and the device is operating in AED mode, and can be given priority over all audible signals except for sounds made to alert the user to defibrillator charging and shock delivery when the device is operating in manual mode.

It will be understood that protocols that include chest compressions but no ventilations are considered CPR protocols, and administration of chest compressions without ventilations under such protocols is considered CPR as used herein.

It will be apparent to those skilled in the art that various modifications and variations can be made to the above-described embodiment(s) of the invention without departing from the spirit and scope of the invention. Thus, it is intended that the present invention cover the modifications and variations of the embodiments provided they come within the scope of the appended claims and their equivalents.

What is claimed is:

1. A method of providing instruction on the performance of chest compressions comprising:
providing a series of signals of a first type corresponding to the desired rhythm of delivery of chest compressions in a chest compression series; and
providing a signal of a second type which indicates a desired point in the first series.

2. The method of claim 1 wherein the desired point is a point near the end of the chest compression series.

3. The method of claim 2 wherein the signal of the second type is a voiced countdown to the end of the chest compression series.

4. The method of claim 2 wherein the signals of the first type are a series of identical sounds delivered in the rhythm desired for the chest compressions and the signal of the second type is a second series of sounds which are distinct from the sounds of the first series and which correspond to the rhythm of the last N compressions in the first series, where N is a predetermined number.

5. The method of claim 4 wherein N is greater than or equal to two.

6. The method of claim 3 wherein the voiced countdown includes the words “two, one” in a rhythm corresponding to the desired rhythm of the last two compression of the series.

7. The method of claim 1 wherein the desired point in the first series includes a first point at a desired interval from the first compression.

8. The method of claim 7 further comprising providing a signal at a second point in the first series at a desired interval from the first point.

9. The method of claim 7 wherein the desired interval is measured in number of compressions.

10. The method of claim 7 wherein the desired interval is measured in time.

11. The method of claim 2 wherein the signals of the first type are tonal signals of a first type, and the signal of the second type is a series of a second kind of tonal signals which are distinguishable from the tonal signals of the first type.

12. The method of claim 2 further comprising providing a prompt which instructs the user to provide ventilation to the patient, the duration of the prompt being at least as long as the desired duration of the ventilation.

13. The method of claim 12 wherein the ventilation prompt includes a voice prompt.

14. The method of claim 12 wherein the ventilation prompt is a sound prompt which approximates the sound of a ventilation bag.

15. The method of claim 1 further comprising the step of choosing between a first prompting protocol appropriate for a patient with a protected airway and a second prompting protocol appropriate for a patient with an unprotected airway.

16. The method of claim 1 further comprising the step, prior to the step of providing a series of signals of a first type, of
detecting delivery of a chest compression, and
commencing delivery of the prompts in response to detecting a chest compression.

17. A method of instructing on delivery of CPR to a patient comprising the steps of:
choosing between a protocol for a patient with a secured airway and a patient with an unsecured airway,
delivering prompts according to the chosen protocol which include rhythmic prompts delivered at the desired rate of chest compressions.

18. The method of claim 17 wherein the choosing step further includes choosing between a protocol for an adult patient and a protocol for a non-adult patient.

19. The method of claim 17 wherein the step of choosing is performed during the delivery of a series of chest compressions.

20. The method of claim 17 further including the step of providing a second series of prompts prior to completion of the chest compression series which provides indication that the end of the chest compression series is nearing.

21. The method of claim 17 further comprising the step, prior to the step of delivering prompts, of detecting delivery of a chest compression, and
commencing delivery of the prompts in response to detecting a chest compression.

22. A device for providing instruction on the performance of chest compressions comprising:
a user interface output device; and
a processor capable of instructing the user interface output device to produce a series of signals of a first type corresponding to the desired rhythm of delivery of chest compressions in a chest compression series and a signal of a second type which indicates a desired point in the first series.

23. The device of claim 23 further comprising a sensor in communication with the processor that detects a parameter indicative of delivery of a chest compression.

24. The device of claim 23, wherein the parameter is patient impedance.

25. The device of claim 24, wherein the sensor includes electrodes adapted to be applied to a patient, and wherein the device further includes an energy storage device electrically coupled to the electrodes.

26. The device of claim 22 further comprising
a memory in which instructions for a plurality of CPR protocols is stored; and
a user interface input in communication with the processor; and
wherein the processor is capable of calling up a CPR protocol from the memory in response to the input information.
